(19) United States
(12) Patent Application Publication
(10) Pub. No.: US 2016/0144171 A1
(43) Pub. Date: May 26, 2016

(54) SYSTEMS AND METHODS FOR GENERATING BIPHASIC WAVEFORMS

(71) Applicant: TrioWave Technologies, Fremont, CA (US)

(72) Inventor: Huiyou Zhu, Fremont, CA (US)

(21) Appl. No.: 14/823,978

(22) Filed: Aug. 11, 2015

Related U.S. Application Data
(60) Provisional application No. 62/084,202, filed on Nov. 25, 2014.

(51) Int. Cl. A61N 1/36; A61N 1/08

(52) U.S. Cl. CPC A61N 1/36; A61N 1/08

(57) ABSTRACT

This present disclosure describes systems that use a constant current source to generate biphasic waveforms. In one example, a positive, constant current is generated from a single constant current source, and then applied to either end of an output workload in an alternating manner, such as at a predefined frequency. In another example, a constant current source is used to generate a positive phase, and a constant current sink along with a voltage source are used to generate a negative phase. An alternating adoption of these two phases, therefore, gives rise to the application of a biphasic waveform on an output workload.

### CROSS-REFERENCE TO RELATED PATENT APPLICATIONS

This application claims the benefit under 35 U.S.C. §119(e) of U.S. Provisional Application 62/084,202, filed Nov. 25, 2014, which is incorporated herein by reference in its entireties and for all purposes.

### BACKGROUND

This disclosure relates to systems and methods for generating biphasic waveforms. Conventionally, biphasic waveforms can be generated with one or two power sources. When generated with just one power source, a biphasic waveform (e.g., sine or square waveforms) can be generated with techniques such as voltage source-based H-bridge. Such techniques, however, require the use of complex circuitry and high power sources.

Portable therapeutic devices such as micro current-based electrotherapy devices usually require the generation of constant currents from a compact device that has limited space for circuits and less powerful power sources. Therefore, voltage source-based techniques that typically include the use of amplifying and shifting components and comparators are not suitable for such portable devices.

There is a need, therefore, to provide systems and methods for generating biphasic waveforms efficiently from a limited power supply and in a small form factor.

### SUMMARY

This present disclosure describes systems that use a constant current source and/or a constant current sink with one power source to generate biphasic waveforms. In one example, a positive, constant current is generated from a single constant current source, and then applied to either end of an output workload in an alternating manner, such as at a predefined frequency.

In another example, a constant current source is used to generate a positive phase, and a constant current sink is used to generate a negative phase. An alternating adoption of these two phases, therefore, gives rise to the application of a biphasic waveform on an output workload.

In accordance with one embodiment of the present disclosure, therefore, provided is a system for generating a biphasic waveform to a workload, comprising: a constant current source configured to generate a constant current; a first switch having a first end and a second end, wherein the first end switches between the current source and the ground; a second switch having a third end and a fourth end, wherein the third end switches between the current source and the ground; and a microcontroller configured to set the first switch and the second switch to alternate between configurations (a) and (b): (a) the first end connects to the current source and the third end connects to the ground; and (b) the first end connects to the ground and the third end connects to the current source, wherein, when a workload is connected to the second end of the first switch and the fourth end of the second switch, configuration (a) allows the constant current to be applied to the workload from the first switch, through the workload and the second switch, to the ground, and configuration (b) allows the constant current to be applied to the workload from the second switch, through the workload and the first switch, to the ground, thereby applying a biphasic waveform with alternating phases to the workload.

Another embodiment of the disclosure provides a system for generating a biphasic waveform to a workload, comprising: a constant current source configured to generate a constant first current; a current sink; a switch having a first end and a second end, wherein the second end switches between a power supply and the ground; and a microcontroller configured to set the current source, the current sink, and the switch to alternate between configurations (a) and (b): (a) the current sink is deactivated or disconnected and the switch connects to the ground; and (b) the current source is deactivated or disconnected and the switch connects to the power supply, wherein, when a workload is connected to the second end of the switch and the current source or the current sink, configuration (a) allows the constant first current to be applied to the workload from the current source, through the workload and the switch, to the ground, and configuration (b) allows the current sink to absorb a second current from the power supply, through the switch and the workload, thereby applying a biphasic waveform with alternating phases to the workload.

### BRIEF DESCRIPTION OF THE DRAWINGS

Provided as embodiments of this disclosure are drawings which illustrate by exemplification only, and not limitation, wherein:

FIG. 1 illustrates a device for generating a biphasic waveform that includes a constant current source; and

FIG. 2 illustrates another device for generating a biphasic waveform, which includes a constant current source and a constant current sink.

It will be recognized that some or all of the figures are schematic representations for exemplification and, hence, that they do not necessarily depict the actual relative sizes or locations of the elements shown. The figures are presented for the purpose of illustrating one or more embodiments with the explicit understanding that they will not be used to limit the scope or the meaning of the claims that follow below.

### DETAILED DESCRIPTION

The present inventions now will be described more fully hereinafter with reference to the accompanying drawings, in which some examples of the embodiments of the inventions are shown. Indeed, these inventions may be embodied in many different forms and should not be construed as limited to the embodiments set forth herein; rather, these embodiments are provided by way of example so that this disclosure will satisfy applicable legal requirements. Like numbers refer to like elements throughout.

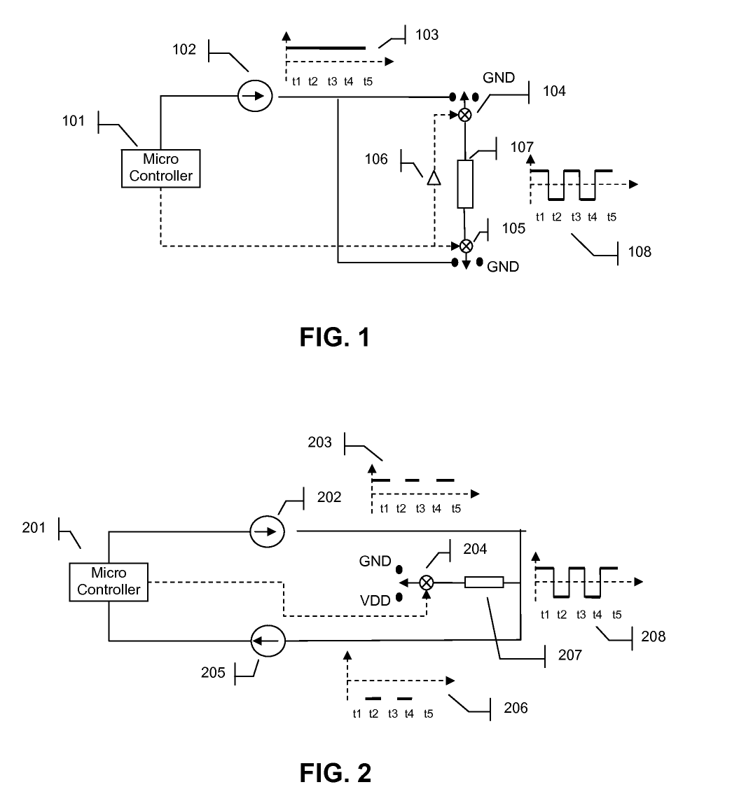

The present disclosure presents two representative designs of devices and methods for generating biphasic waveforms, as illustrated in FIG. 1 and FIG. 2.

As used herein, a “biphasic waveform” is a current waveform that includes primarily two phases appearing in an alternating manner. In one aspect, one of the phases is positive and the other negative. In another aspect, both phases are positive or negative, but one has a higher amplitude than the other. In one aspect, both phases are constant or substantially constant. In another aspect, the phases are asymmetric such as one with a positive of square wave and a negative in sine wave. In another aspect, the phases are symmetric such as sine waves.

In a particular embodiment, the biphasic waveform comprises a constant positive phase and a constant negative phase having the same or substantially the same amplitudes. Nevertheless, it is readily appreciated that the devices and methods of the present technology, such as those illustrated in FIGS. 1 and 2 and further described below, can be used to generate any biphasic waveforms, including symmetric and asymmetric waveforms.

With reference to FIG. 1, in one embodiment, the device of the present disclosure includes a microcontroller 101, a current source 102, two control switches 104 and 105, and optionally a convertor 106. In some instances, the current source is a constant current source, generating positive, constant current.

A “current source” is an electronic circuit that delivers an electric current which is independent of the voltage across it. A current source can be an independent current source that delivers a constant current. A dependent current source, by contrast, delivers a current which is proportional to some other voltage or current in the circuit.

Current sources are different from voltage sources. A theoretical voltage source would have a zero ohm output impedance in series with the source. A real-world voltage source has a very low, but non-zero output impedance: often much less than 1 ohm. By contrast, a current source provides a constant current, as long as the load connected to the source terminals has sufficiently low impedance. An ideal current source would provide no energy to a short circuit and approach infinite energy and voltage as the load resistance approaches infinity (an open circuit). An ideal current source has an infinite output impedance in parallel with the source. A real-world current source has a very high, but finite output impedance.

Microcontroller 101 can control the operation of constant current source 102, such as sending a digital waveform to the constant current source or turn it on or off. Further, microcontroller 101 regulates control switches 104 and 105, optionally through controlling convertor 106 that converts digital control signal from microcontroller 101 to the control pins of switches 104 and 105.

Switches 104 and 105, for instance, can be multiplexer (or mux) type switches. Each switch can include a control pin that is controlled by microcontroller 101. Switch 104 has two terminals, one connected to an output workload when in use, and the other switching between the output terminal of current source 102 and the ground. Likewise, switch 105 has two terminals, one connected to an output workload when in use (the opposite side from the terminal of switch 104), and the other switching between the output terminal of current source 102 and the ground.

When in operation, the device is connected to an output workload 107, through two terminals of switches 104 and 105. Microcontroller 101 controls switches 104 and 105 to set in two different configurations in an alternating manner.

In configuration (a), switch 104 connects with current source 102 and switch 105 connects to the ground. Therefore, in this configuration, the current from current source 102 goes through switch 104, workload 107, switch 105, and to the ground.

In configuration (b), switch 104 connects to the ground and switch 105 connects with current source 102. Therefore, in this configuration, the current from current source 102 goes through switch 105, workload 107, switch 104, and to the ground.

When microcontroller 101 controls the switches 104 and 105 to alternate between configurations (a) and (b), therefore, workload 107 receives the current from two opposite directions, resulting in an application of a biphasic waveform on workload 107.

The alternation of the phases can take place at a pre-determined schedule, such as at a predefined frequency. For instance, as shown in FIG. 1, the system is set at configuration (a) at time slots t1 and t3 and at configuration (b) at time slots t2 and t4, leading to the generation of a biphasic waveform 108 that has positive phases at time slots t1 and t3 and negative phases at time slots t2 and t4.

With reference to FIG. 2, in another embodiment, the device of the present disclosure includes a microcontroller 201, a current source 202, a control switch 204, a positive supply voltage (VDD), and a current sink 205. In some instances, the current source is a constant current source, generating positive, constant current. In some instances, the current sink is a constant current sink, that generates a constant current by absorbing currents from the VDD.

Microcontroller 201 can control the operation of current source 202 and current sink 205. Further, microcontroller 201 regulates switch 204.

Switch 204, for instance, can be multiplexer (or mux) type switches. The switch can include a control pin that is controlled by microcontroller 201. Switch 204 has two terminals, one connected to an output workload when in use, and the other switching between the ground and the VDD.

When in operation, the device is connected to an output workload 207 which, at one end, connects to current source 202 and current sink 205, and at the other end, connects to switch 204. Accordingly, microcontroller 201 can control switch 204 to set in two different configurations in an alternating manner.

In configuration (a), switch 204 connects with the ground. Optionally, at this configuration, microcontroller 201 turns off current sink 205 and/or voltage supply VDD. Therefore, in this configuration, the current from current source 202 goes through workload 207 and switch 204, and to the ground.

In configuration (b), switch 204 connects to the voltage supply VDD. Optionally, at this configuration, microcontroller 201 turns off current source 202. Therefore, in this configuration, a current arises from VDD, going through workload 207 into current sink 205.

When microcontroller 201 controls the switch 204 to alternate between configurations (a) and (b), therefore, workload 207 either receives a current from current source 202 or from voltage source 207 absorbed by the current sink. These currents come from two opposite directions, resulting in an application of a biphasic waveform on workload 207.

The alternation of the phases can take place at a pre-determined schedule, such as at a predefined frequency. For instance, as shown in FIG. 2, the system is set at configuration (a) at time slots t1 and t3 and at configuration (b) at time slots t2 and t4, leading to the generation of a biphasic waveform 208 that has positive phases at time slots t1 and t3 and negative phases at time slots t2 and t4.

The biphasic waveforms illustrated in FIGS. 1 and 2 are square waveforms. The present technology, however, is able to generate generic biphasic waveforms. For instance, different current sources or sinks can be used to generate waveforms of different variety. Alternatively, once the biphasic waveforms are generated, they can further be modified to suit a particular need.

For instance, the biphasic waveforms of the present disclosure can serve as the base waveform for generating a therapeutic waveform that includes overshot signals, as further described in detail in U.S. Provisional Application No. 62/037,029, filed Aug. 13, 2014, entitled “Overshoot Waveform in Micro Current Therapy.” The content of the application is incorporated into the present disclosure in its entirety, by reference.

The amplitudes of the currents generated by the current sources can be adjusted as needed. For instance, for therapeutic use, the amplitude can in general be from 1 μA to 200 μA, or alternatively from 5 μA to 100 μA, from 20 μA to 80 μA, from 30 μA to 60 μA or from 30 μA to 50 μA.

The frequency of the biphasic waveform can also be adjusted depending on needs. For instance, the frequency (i.e., the alternating frequency) can be from 0.1 Hz to 200 Hz. In some aspects, the frequency is greater than 0.1 Hz, 0.2 Hz, 0.5 Hz, 1 Hz, 2 Hz, 5 Hz, 10 Hz or 20 Hz. In some aspects, the frequency is lower than 200 Hz, 180 Hz, 150 Hz, 100 Hz, 90 Hz, 80 Hz, 70 Hz, 60 Hz, 50 Hz, 40 Hz, 30 Hz, 20 Hz, 10 Hz or 5 Hz.

The waveforms of the present disclosure can be used to generate voltage potentials suitable for stimulating excitable cells in a human body so that the cell enters a polarization stage. To trigger a cell like a nerve cell to enter such stage, a voltage potential change has to be large enough to reach a threshold (usually between −20 mV and −65 mV depending on type of cell or nerve).

It is contemplated that the biphasic waveforms generated by the present technology can be used for micro current therapies, in particular for home use. Accordingly, methods of micro current electrotherapy are also provided, in some embodiments. The output waveforms that can be generated from the systems are applied to a human subject. Various parameters of the waveforms can be adjusted to suit the user or the particular disease or condition that the user has, such as back pain, arthritis at the knee, or wound on the skin.

A micro current electrotherapy entails sending relatively weak electrical signals into the body of an individual in need of the therapy. Such therapies apply small (e.g., between 1 and 50 microampere) electrical currents to nerves using electrodes placed on the skin. Micro current electrotherapies can be used in treatments for pain, age-related macular degeneration, wound healing, and tendon repair, without limitation. Many micro current treatments concentrate on pain and/or speeding healing and recovery. Micro current treatments are commonly used by professional and performance athletes with acute pain and/or muscle tenderness as they are drug-free and non-invasive, thus avoiding testing and recovery issues. They can also be used as a cosmetic treatment.

### Overshoot Waveforms Based on the Biphasic Waveforms

The biphasic waveforms that can be generated from the systems and methods of the present disclosure can serve as base waveforms for generating other waveforms, such as overshoot waveforms useful for micro current therapies. A non-limiting system and method for generating and using overshoot waveforms is described in U.S. patent application Ser. No.______/______,______ (Atty. Dkt. No. 41HA-201816-US), entitled “Overshoot Waveform in Micro Current Therapy,” filed Aug. 11, 2015, which claims benefit under 35 U.S.C. §119(e) of U.S. Provisional Application 62/037,029, filed Aug. 13, 2014. The content of each of these applications is incorporated to the present disclosure by reference in its entirety.

An overshoot waveform includes overshoot signals overlaid on constant currents and is capable of stimulating cells or nerves to achieve healing. In one embodiment, the system includes an oscillator configured to generate a reference signal, a pulse wave generator to generate a pulse waveform signal based on the reference signal, an overshoot generator to generate an overshoot signal based on the reference signal, and an output module to generate a composite output waveform signal based on the overshoot signal and the pulse waveform signal, wherein the composite output waveform includes one or more pulses having one or more overshoots that extend a width of each of the pulses. The system can be adjusted by a user depending on the disease, condition, or preference of the user.

A square waveform (or more generally a pulse waveform) can incorporate overshoot signals that improve the ability to stimulate cells to achieve therapeutic benefits. The base pulse waveform can be conventional pulse waveform and can be mono-phasic or biphasic. Each phase can be substantially constant, that is, the variation (e.g., the standard deviation of the amplitude within each phase) is within 10% (or 5%) of the amplitude.

The amplitude of the base pulse waveform can be adjusted, and is in general from 1 μA to 200 μA, or alternatively from 5 μA to 100 μA, from 20 μA to 80 μA, from 30 μA to 60 μA or from 30 μA to 50 μA. In one aspect, the overshoot signal can have a rapid rising phase that could go over threshold potential for cell or nerve polarization. The overshoot signal can have a slow falling phase and resting phase of overshoot signal and decay into base pulse wave stage. Overshoot signal can occur multiple times (e.g., at least 2, at least 3, at least 4, at least 5 times) in a single phase with a frequency resonating with the type of cell and nerve.

The frequency of the base pulse wave can also be adjusted, for instance, by a user through an input module. In general, the frequency is from 0.1 Hz to 200 Hz. In some aspects, the frequency is greater than 0.1 Hz, 0.2 Hz, 0.5 Hz, 1 Hz, 2 Hz, 5 Hz, 10 Hz or 20 Hz. In some aspects, the frequency is lower than 200 Hz, 180 Hz, 150 Hz, 100 Hz, 90 Hz, 80 Hz, 70 Hz, 60 Hz, 50 Hz, 40 Hz, 30 Hz, 20 Hz, 10 Hz or 5 Hz.

The base pulse wave can be bisphasic or mono-phasic waveforms. In some aspects, the base pulse wave is a square wave.

The overshoot waveforms can be used to generate voltage potentials suitable for stimulating excitable cells in a human body so that the cell enters a polarization stage. To trigger a cell like a nerve cell to enter such stage, a voltage potential change has to be large enough to reach a threshold (usually between −20 mV and −65 mV depending on type of cell or nerve).

An overshoot signal that is suitable for generating a composite outcome waveform can have a rising edge (left of the peak) that is steeper than a falling edge (right of the peak). The rising edge, in some aspects, reaches the peak within 2 ms, or alternatively within 0.1 ms, 0.2 ms, 0.3 ms, 0.4 ms, 0.5 ms, 0.6 ms, 0.7 ms, 0.8 ms, 0.9 ms, 1 ms, 1.2 ms, 1.5 ms, or 1.8 ms.

In some aspects, the failing edge shows a plateau, which can last from 0.5 ms to 10 ms. In some aspects, the time span of the falling edge (from peak to back to a pulse phase) is at least twice (or alternatively 3×, 4×, 5×, 10×) the time span of the rising edge (from a pulse phase to peak). The gradual falling of the overshoot amplitudes helps to achieve the resting state of polarization and avoid another threshold triggering before the previous polarization stage is completed.

In some aspects, there is a resting period between each overshoot signal. In one aspect, the resting period is at least 0.5 ms, or alternatively at least 1 ms, 1.5 ms, 2 ms, 3 ms, 4 ms or 5 ms. In one aspect, the resting period is not longer than 20 ms, 10 ms, 5 ms, 4 ms, 3 ms or 2 ms. In one aspect, the resting period is at least 50% of the time span of the overshoot signal, or alternatively at least 75%, 100%, 150%, 2×, 3×, 4× or 5× of the time span of the overshoot signal. In one aspect, the resting period is not longer than 2×, 3×, 4×, 5×, 10× or 20× of the time span of the overshoot signal.

The frequency, shape and amplitude of the overshoot signals can be adjusted. The peak amplitude, in general, is adequate to genera a 0 to +/−100 mV voltage potential, and in some aspects between −20 mV and −65 mV depending on type of cell or nerve. In some aspects, the amplitude of the overshoot signal is between 1 μA and 600 μA. In one aspect, the amplitude of the overshoot signal is greater than 1 μA, 2 μA, 3 μA, 4 μA, 5 μA, or 10 μA. In one aspect, the amplitude of the overshoot signal is less than 500 μA, 400 μA, 300 μA, 200 μA, 100 μA, 90 μA, 80 μA, 70 μA, 60 μA, 50 μA, 40 μA, 30 μA, 20 μA, or 10 μA. In some aspects, the amplitude of the overshoot signal is at least 10%, or alternatively at least 20%, 30%, 40%, 50%, 75%, 100%, 150%, 200% or 250% of the amplitude of the base pulse signal. In some aspects, the amplitude of the overshoot signal is not greater than 10 times, 9 times, 8 times, 7 times, 6 times, 5 times, 4 times, 3 times, or twice of the amplitude of the base pulse signal.

The frequency of the overshoot signals can be greater than that of the base pulse waveform, so that each phase of the base pulse waveform is superimposed (or overlaid) with at least an overshoot signal. In one aspect, the frequency is at least 1 Hz, or alternatively at least 2 Hz, 5 Hz, 10 Hz, 15 Hz, 20 Hz, 30 Hz, 40 Hz, 50 Hz, 100 Hz, 150 Hz or 200 Hz. In some aspects, the frequency is not greater than 5 KHz, 2 KHz, 1 KHz, 500 Hz, 200 Hz, 150 Hz or 100 Hz.

In some aspects, the frequency, amplitude, shape, resting period, of any of the above waveforms or signals can be adjusted on the fly during a therapy. The adjustment can be automatic or triggered by input from a user.

An example system useful for generating an overshoot waveform is also provided, which can include an Overshoot Generator that represents a micro-controller based component that is programmable to produce digitized overshoot signal according to requested parameters, and output digitized overshoot. Also included is a Pulse Wave Generator that is a programmable component that takes requested parameters to generate digitized pulse wave signal. Both the Overshoot Generator and the Pulse Wave Generator are connected to an Oscillator for signal synchronization. Both digitized signals can be sent to a composition component for composition, and sent to digital-to-analog converter (DAC)/Shifting to convert into analog signal.

An oscillator herein is an electronic circuit that synchronizes the generation of pulse waves and overshoot waves in a digital format. A digital-to-analog converter (DAC) is a function that converts digital data (usually binary) into an analog signal (current, voltage, or electric charge). An analog-to-digital converter (ADC) performs the reverse function.

In some aspects, the system further includes wires and/or electrodes to connect to the skin or other organs of a human subject so as to apply an output waveform to the user. in some aspects, the system further includes an input module or device that takes an input from the user. The input can then be used to start, stop or adjust the waveform applied to the user. In one aspect, the input module is wired to the system and in another aspect, the input module communicates with the system wirelessly. In some aspects, the input module includes a graphic user interface. In some aspects, the system includes a processor to take the input and implement adjustments.

Methods of micro current electrotherapy are also provided, in some embodiments. The output waveforms that can be generated from the systems are applied to a human subject. Various parameters of the waveforms can be adjusted to suit the user or the particular disease or condition that the user has, such as back pain, arthritis at the knee, or wound on the skin.

A micro current electrotherapy entails sending relatively weak electrical signals into the body of an individual in need of the therapy. Such therapies apply small (e.g., between 1 and 50 microampere) electrical currents to nerves using electrodes placed on the skin. Micro current electrotherapies can be used in treatments for pain, age-related macular degeneration, wound healing, and tendon repair, without limitation. Many micro current treatments concentrate on pain and/or speeding healing and recovery. Micro current treatments are commonly used by professional and performance athletes with acute pain and/or muscle tenderness as they are drug-free and non-invasive, thus avoiding testing and recovery issues. They can also be used as a cosmetic treatment.

### Progressive Signal Adjustment

When using the biphasic waveforms (or overshoot waveforms based on biphasic waveforms) for a micro current therapy, it can be helpful to apply progressive signal adjustment. Battery-driven stimulators for electrotherapy usually equip with a small battery to generate specified current waveforms applied to human body for stimulation. Such current waveforms can easily get into a saturated status due to high bio-impedance and low battery power. The present disclosure also provides systems and methods that overcome this problem. In one embodiment, the waveform (e.g., the biphasic waveforms or the overshoot waveforms) is adjusted according to feedbacks taken from a patient to reduce or avoid saturation. In another embodiment, the system includes a detachable battery pack that enables convenient use and charging of a battery in the battery pack.

Further details of such system and method are provided in U.S. patent application Ser. No. ______/______,______ (Atty. Dkt. No. 41HA-201815-US), entitled “Signal Adjustment for Electrotherapy,” filed Aug. 11, 2015, which claims benefit under 35 U.S.C. §119(e) of U.S. Provisional Application 62/043,894, filed Aug. 29, 2014, and U.S. patent application Ser. No.______/______,______ (Atty. Dkt. No. 41HA-202942-US), entitled “Battery Pack for Electrotherapy Devices,” filed Aug. 11, 2015, which claims benefit under 35 U.S.C. §119(e) of U.S. Provisional Application 62/043,912, filed Aug. 29, 2014. The content of each of these applications is incorporated to the present disclosure by reference in its entirety.

For a battery that has a preset working voltage range, the current of its output waveforms is restricted by bio-impedance that usually is high, such as 200 KOhm or above, at an early stage, and gradually comes down to an acceptable level, 2 KOhm or less, after minutes or hours. This bio-impedance is carried by electrodes that apply stimulation signals to human body to gradually establish ionic channels to reduce bio-impedance.

A conventional electrotherapy device typically does not take the influence of the bio-impedance into consideration, but simply generates a predetermined waveform that can result in a saturated output waveform (i.e., exceeding working power range), or an invalid output waveform for electrotherapy.

It is discovered by the instant inventor that progressive adjustment of a current waveform can be used to reduce or even prevent occurrence of saturated waveform outputs. The adjustment can take the voltage between the electrodes as input when the electrodes are applied on the skin or body of a human patient. The voltage can be directly measured or derived from other parameters, as further described below.

One objective of the adjustment is to gradually increase the voltage within a suitable range at an appropriate pace. For instance, if the determined voltage is below the upper limit of the range, the system then increases the amplitude of the waveform output by a certain interval. The voltage determination can be repeated at a desired frequency, until the voltage reaches or exceeds the upper limit, at which point the amplitude is reduced to a base level. Afterwards, the voltage determination can continue; so will the increase of the amplitude after each determination.

An example electrical circuit suitable for implementing such progressive adjustment includes a micro controller that includes at least a waveform generator such as a pulse wave generator (e.g., a square wave generator), a processor, and memory that embeds program code for carrying out desired control of the waveform generator. The micro controller is connected to two electrodes (first and second) that output generated current waveforms to a bio impedance (e.g., in a patient body). The current waveform path, as shown, starts from the micro controller, the first electrode, the bio impedance to the second electrode, and back to the micro controller. A analog-to-digital convertor (ADC) can take instruction from the micro controller to gather voltage potentials from the electrodes.

A. Initialization

A 1^{st} step of the progressive adjustment initializes the process at which point the following parameters can be set: (a) voltage determination schedule (e.g., a constant time interval), (b) voltage upper limit, (c) maximum and minimum amplitudes, and (d) amplitude increase interval.

The voltage determination schedule, in one aspect, can be a fixed schedule such as repeating the determination at a constant time interval. In another aspect, the interval can increase or decrease where needed. In yet another aspect, the schedule includes a rule for setting the schedule on the fly. For instance, the schedule can be adjusted depending on the determined voltage. For example, when the voltage is close to the upper limit, the determination can be carried out more frequently.

The voltage upper limit can be a fixed value, or set with a user profile, user preference, user input or by the system. Likewise, the maximum and minimum amplitudes for the waveform output can initialized. The amplitude increase interval can be a constant number or determined according to the voltage upper limit, and/or the maximum and minimum amplitudes.

The amplitude of the waveform can have a range from 1 μA to 100 mA, in some aspects. Alternatively, the minimum amplitude can be 2 μA, 3 μA, 4 μA, 5 μA, 10 μA, 20 μA, 30 μA, 50 μA or 100 μA. In some aspects, the maximum amplitude can be 100 μA, 150 μA, 200 μA, 250 μA, 300 μA, 400 μA, 500 μA, 1 mA, 5 mA, 10 mA, 20 mA, 50 mA, 60 mA or 100 mA.

In some aspects, the waveform has a frequency from 0.1 Hz to 200 Hz. In one aspect, the frequency is at least 0.1 Hz, or at least 0.2 Hz, 0.5 Hz, 1 Hz, 5 Hz, 10 Hz or 20 Hz. In one aspect, the frequency is not higher than 500 Hz, 400 Hz, 300 Hz, 200 Hz, 100 Hz, or 50 Hz.

In some aspects, the time interval is at least 5 seconds, or at least 10, 20, 30, 40, 50, or 60 seconds. In some aspects, the time interval is not longer than 30 seconds, or not longer than 60 seconds, 2 minutes, 3 minutes, 4 minutes, 5 minutes or 10 minutes.

In some aspects, the amplitude increase interval is at least 1 μA, or alternatively at least 2 μA, 3 μA, 4 μA, 5 μA, 10 μA, 20 μA, 50 μA, or 100 μA. In some aspects, the amplitude increase interval is not greater than 100 μA, 150 μA, 200 μA, 250 μA, 300 μA, 400 μA, 500 μA, 1 mA, 5 mA, or 10 mA.

In some aspects, the voltage upper limit is at least 1000 mV, 2000 mV, 3000 mV, 4000 mV, 5000 mV. In some aspects, the voltage upper limit is not higher than 5000 mV, 7000 mV, 9000 mV, 10,000 mV, 12,000 mV or 15,000.

At the 2^{nd} step, the waveform generator generates a current waveform with the minimum amplitude.

B. Voltage Determination

At the 3^{rd} step, the voltage between the electrodes can be determined by ADC sampling, which represents a product of current waveform and bio-impedance. Alternatively, the voltage can be computed from the amplitude and ADC sampling of the bio-impedance.

C. Adjustment

At the 4^{th} step, the determined voltage is compared to the voltage upper limit. If the voltage is close to, equal to, or over the upper limit, then the system adjusts the waveform generation to decrease its output's amplitude (5^{th} step). In one aspect, the amplitude is decreased to a base amplitude level (e.g., the minimum amplitude set at the 1^{st} step).

If the voltage is lower than the upper limit, then the system adjusts the waveform generation to increase its output's amplitude (8^{th} step) by the increase interval set at the step. In some aspects, a 7^{th} step is included to ensure that the amplitude does not exceed the maximum amplitude allowed by the system. Due to unpredictable changes of bio-impedance from electrodes, continuous monitoring of the current waveform is preferred to ensure that voltage is in a desired working range. In either scenario, the system will continue to monitor the voltage (6^{th} step).

The voltage upper limit, in some aspects, can be dynamically changed. For instance, at the 4^{th} step, the microcontroller can measure the battery voltage range from the battery pack. Then, the measured battery voltage range can be used to adjust the voltage upper limit. For instance, if the measured battery voltage range has shifted downwards, then the voltage upper limit can be reduced too, by, e.g., 0.05, 0.1 or 0.2 volt, or by certain percentage (e.g., 1%, 2%, 5%, 10%).

### Battery Pack

Another embodiment of the present disclosure provides an electrotherapy device that includes a battery pack which is detachably connected to a stimulation pack that includes a waveform generator and control circuits. Without limitation, the stimulation pack can include a microprocessor, digital and analog circuits such as waveform generator, memory, IO pin, oscillator, and/or ADC and DAC.

In one embodiment, the electrotherapy device includes two detachably connected packs (or enclosures), a battery pack and a stimulation pack. The stimulation pack contains at least an electric signal (waveform) generator configured to generate an electric signal and a controller configured to control the generation of the electric signal. The stimulation pack can be connected to a first electrode and to a first connector each of which is in electric communication with the signal generator.

The battery pack can contain at least a battery holder for holding a battery, which holder includes a positive contact and a negative contact. The battery pack, in one aspect, further includes a charging and protection circuit configured to prevent overdrain and overcharge of the battery and a battery indicator configured to indicate a status of the battery. Like the stimulation pack, the battery pack can also be connected to a second electrode and to a second connector each in electric communication with the battery or the charging and protection circuit. In some embodiments, the device further includes a battery, such as a rechargeable battery, in the battery holder.

The first connector and the second connector can be detachably connected to enable electric communication between the signal generator and the charging and protection circuit. Further, the first electrode and the second electrode have a maximum distance of at least 25 cm (or 30 cm, 40 cm, 50 cm, 100 cm, 200 cm, 250 cm, or 300 cm) such that they can be placed on different locations of the body of a patient.

In some aspects, each of the packs can be connected to the corresponding connector through an electric wire. In one aspect, one of the connectors can be directly disposed on the pack.

Although the discussions above may refer to a specific order and composition of method steps, it is understood that the order of these steps may differ from what is described. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure. Such variations will depend on the software and hardware systems chosen and on designer choice. It is understood that all such variations are within the scope of the disclosure. Likewise, software and web implementations of the present disclosure could be accomplished with standard programming techniques with rule based logic and other logic to accomplish the various database searching steps, correlation steps, comparison steps and decision steps.

Unless otherwise defined, all technical and scientific terms used herein have the same meaning as commonly understood by one of ordinary skill in the art to which this disclosure belongs.

The disclosures illustratively described herein may suitably be practiced in the absence of any element or elements, limitation or limitations, not specifically disclosed here. For example, the terms “comprising”, “including,” containing,” etc. shall be read expansively and without limitation. Additionally, the terms and expressions employed here have been used as terms of description and not of limitation; hence, the use of such terms and expressions does not evidence and intention to exclude any equivalents of the features shown and described or of portions thereof. Rather, it is recognized that various modifications are possible within the scope of the disclosure claimed.

By the same token, while the present disclosure has been specifically disclosed by preferred embodiments and optional features, the knowledgeable reader will apprehend modification, improvement and variation of the subject matter embodied here. These modifications, improvements and variations are considered within the scope of the disclosure.

The disclosure has been described broadly and generically here. Each of the narrower species and subgeneric groupings falling within the generic disclosure also form part of the disclosure. This includes the generic description of the disclosure with a proviso or negative limitation removing any subject matter from the genus, regardless of whether or not the excised material is described specifically.

Where features or aspects of the disclosure are described by reference to a Markush group, the disclosure also is described thereby in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Although the disclosure has been described in conjunction with the above-mentioned embodiments, the foregoing description and examples are intended to illustrate and not limit the scope of the disclosure. Other aspects, advantages and modifications within the scope of the disclosure will be apparent to those skilled in the art to which the disclosure pertains.

1. A system for generating a biphasic waveform to a workload, comprising:
a constant current source configured to generate a constant current;
a first switch having a first end and a second end, wherein the first end switches between the current source and the ground;
a second switch having a third end and a fourth end, wherein the third end switches between the current source and the ground; and
a microcontroller configured to set the first switch and the second switch to alternate between configurations (a) and (b):
(a) the first end connects to the current source and the third end connects to the ground; and
(b) the first end connects to the ground and the third end connects to the current source,
wherein, when a workload is connected to the second end of the first switch and the fourth end of the second switch, configuration (a) allows the constant current to be applied to the workload from the first switch, through the workload and the second switch, to the ground, and configuration (b) allows the constant current to be applied to the workload from the second switch, through the workload and the first switch, to the ground,
thereby applying a biphasic waveform with alternating phases to the workload.

2. The system of claim 1, wherein the microcontroller is further configured to generate a digital waveform as input to the current source.

3. The system of claim 1, further comprising a converter that implements the configurations based on instructions from the microcontroller.

4. The system of claim 1, wherein the current is positive.

5. The system of claim 1, further comprising a first electrode connected to the second end of the first switch and a second electrode connected to the fourth end of the second switch.

6. The system of claim 1, wherein the alternation has a predetermined frequency.

7. A system for generating a biphasic waveform to a workload, comprising:
a constant current source configured to generate a constant first current;
a current sink;
a switch having a first end and a second end, wherein the second end switches between a power supply and the ground; and
a microcontroller configured to set the current source, the current sink, and the switch to alternate between configurations (a) and (b):
(a) the current sink is deactivated or disconnected and the switch connects to the ground; and
(b) the current source is deactivated or disconnected and the switch connects to the power supply,
wherein, when a workload is connected to the second end of the switch and the current source or the current sink, configuration (a) allows the constant first current to be applied to the workload from the current source, through the workload and the switch, to the ground, and configuration (b) allows the current sink to absorb a second current from the power supply, through the switch and the workload,
thereby applying a biphasic waveform with alternating phases to the workload.

8. The system of claim 7, wherein the microcontroller is further configured to generate a digital waveform as input to the current source.

9. The system of claim 7, wherein the current is positive.

10. The system of claim 7, further comprising a first electrode connected the current source or the current sink and a second electrode connected to the switch.

11. The system of claim 7, wherein the alternation has a predetermined frequency.
